(19) United States
(12) Patent Application Publication
(10) Pub. No.: US 2012/0095521 A1
(43) Pub. Date: Apr. 19, 2012

(54) DETECTION OF HEART RHYTHM USING AN ACCELEROMETER

(75) Inventor: William J. Hintz, Ham Lake, MN (US)

(73) Assignee: MEDTRONIC, INC., Minneapolis, MN (US)

(21) Appl. No.: 12/907,481

(22) Filed: Oct. 19, 2010

(51) Int. Cl. A61N 1/08

(52) U.S. Cl. 607/28

(57) ABSTRACT

Various techniques for using an accelerometer to detect cardiac contractions are described. One example method described includes filtering a signal received by an electrical sensing channel of an implantable medical device (IMD) configured to detect electrical depolarizations of a heart of a patient, identifying a failure of the electrical sensing channel of the IMD based on the filtered signal and, in response to identifying the failure, initiating a mechanical sensing channel of the implantable medical device to identify mechanical cardiac contractions.

### TECHNICAL FIELD

This disclosure relates to medical devices and, more particularly, to medical devices that monitor heart rhythms.

### BACKGROUND

A variety of medical devices for delivering a therapy and/or monitoring a physiological condition have been used clinically or proposed for clinical use in patients. Examples include medical devices that deliver therapy to and/or monitor conditions associated with the heart, muscle, nerve, brain, stomach or other organs or tissue. Some therapies include the delivery of electrical signals, e.g., stimulation, to such organs or tissues. Some medical devices may employ one or more elongated electrical leads carrying electrodes for the delivery of therapeutic electrical signals to such organs or tissues, electrodes for sensing intrinsic electrical signals within the patient, which may be generated by such organs or tissue, and/or other sensors for sensing physiological parameters of a patient.

Medical leads may be configured to allow electrodes or other sensors to be positioned at desired locations for delivery of therapeutic electrical signals or sensing. For example, electrodes or sensors may be carried at a distal portion of a lead. A proximal portion of the lead may be coupled to a medical device housing, which may contain circuitry such as signal generation and/or sensing circuitry. In some cases, the medical leads and the medical device housing are implantable within the patient. Medical devices with a housing configured for implantation within the patient may be referred to as implantable medical devices.

Implantable cardiac pacemakers or cardioverter-defibrillators, for example, provide therapeutic electrical signals to the heart, e.g., via electrodes carried by one or more implantable medical leads. The therapeutic electrical signals may include pulses for pacing, or shocks for cardioversion or defibrillation. In some cases, a medical device may sense intrinsic depolarizations of the heart, and control delivery of therapeutic signals to the heart based on the sensed depolarizations. Upon detection of an abnormal rhythm, such as bradycardia, tachycardia or fibrillation, an appropriate therapeutic electrical signal or signals may be delivered to restore or maintain a more normal rhythm. For example, in some cases, an implantable medical device may deliver pacing stimulation to the heart of the patient upon detecting tachycardia or bradycardia, and deliver cardioversion or defibrillation shocks to the heart upon detecting fibrillation.

Leadless cardiac devices, such as leadless pacemakers, may also be used to sense intrinsic depolarizations and/or other physiological parameters of the heart and/or deliver therapeutic electrical signals to the heart. A leadless cardiac device may include one or more electrodes on its outer housing to deliver therapeutic electrical signals and/or sense intrinsic depolarizations of the heart. Leadless cardiac devices may be postioned within or outside of the heart and, in some examples, may be achored to a wall of the heart via a fixation mechanism.

### SUMMARY

In general, this disclosure describes techniques for using an accelerometer to detect cardiac contractions. An electrical sensing channel may detect a signal indicative of cardiac contractions. If the electrical sensing channel fails, an accelerometer may be activated in response to the failure to provide mechanical redundancy for detecting cardiac contractions. For example, a sensing integrity module may identify a failure of the electrical sensing channel, and in response to the identified failure, a processor may initiate a mechanical sensing channel. Once initiated, the mechanical sensing channel may analyze an accelerometer signal to identify cardiac contractions.

The accelerometer may be positioned within or proximate to a heart of a patient such that it detects the rhythmic motion of one or more walls of the patient's heart. For example, the accelerometer may be positioned within an implantable medical device, such as a leadless pacemaker. A leadless pacemaker may be attached to a wall of the patient's heart, e.g., epicardially or endocardially. As another example, the accelerometer may be positioned within a lead, e.g., proximate to a distal end of a lead positioned within or outside a chamber of the heart. In general, the accelerometer may detect a signal indicative of the rhythmic motion of the heart.

In one example, the disclosure is directed to a method comprising filtering a signal received by an electrical sensing channel of an implantable medical device (IMD) configured to detect electrical depolarizations of a heart of a patient, identifying a failure of the electrical sensing channel of the IMD based on the filtered signal and, in response to identifying the failure, initiating a mechanical sensing channel of the implantable medical device to identify mechanical cardiac contractions.

In another example, the disclosure is directed to a system comprising an accelerometer positioned proximate to a wall of a heart of a patient, an electrical sensing channel configured to detect electrical depolarizations of the heart of the patient, a mechanical sensing channel configured to analyze a signal from the accelerometer to identify mechanical contractions of the heart of the patient, a sensing integrity module configured to filter a signal received by the electrical sensing channel and identify a failure of the electrical sensing channel based on the filtered signal, and a processor configured to initiate the mechanical sensing channel in response to the identified failure.

In another example, the disclosure is directed to a computer-readable medium containing instructions. The instructions cause a programmable processor to filter a signal received by an electrical sensing channel of an implantable medical device (IMD) configured to detect electrical depolarizations of a heart of a patient, identify a failure of the electrical sensing channel of the IMD based on the filtered signal and, in response to identifying the failure, initiating a mechanical sensing channel to identify mechanical cardiac contractions.

In another example, the disclosure is directed to a system comprising means for filtering a signal received by an electrical sensing channel of an implantable medical device (IMD) configured to detect electrical depolarizations of a heart of a patient, means for identifying a failure of the electrical sensing channel of the IMD based on the filtered signal, and means for initiating a mechanical sensing channel to identify mechanical cardiac contractions in response to identifying the failure.

The details of one or more aspects of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example therapy system comprising a leadless implantable medical device (IMD) that may be used to monitor one or more physiological parameters of a patient and/or provide therapy to the heart of a patient.

FIG. 2 is a conceptual diagram illustrating another example therapy system comprising an IMD coupled to a plurality of leads that may be used to monitor one or more physiological parameters of a patient and/or provide therapy to the heart of a patient.

FIG. 3 is a conceptual diagram illustrating the leadless IMD of FIG. 1 in further detail.

FIG. 4 is a conceptual diagram further illustrating the IMD and leads of the system of FIG. 2 in conjunction with the heart.

FIG. 5 is a conceptual drawing illustrating the IMD of FIG. 2 coupled to a different configuration of implantable medical leads in conjunction with the heart.

FIG. 6 is a functional block diagram illustrating an example configuration of an IMD.

FIG. 7 is a block diagram of an example external programmer that facilitates user communication with the IMD.

FIG. 8 is a block diagram illustrating an example system that includes an external device, such as a server, and one or more computing devices that are coupled to the IMD and programmer via a network.

FIG. 9 is a flow diagram of an example method of using an accelerometer to identify cardiac contractions in response to detecting the failure of an electrical sensing channel.

### DETAILED DESCRIPTION

In general, this disclosure describes techniques for using an accelerometer to detect cardiac contractions. Typically, an electrical sensing channel may sense intrinsic depolarizations of the heart, which are indicative of cardiac contractions. If the electrical sensing channel fails, an accelerometer may provide mechanical redundancy for detecting cardiac contractions. For example, a sensing integrity module may identify a failure of the electrical sensing channel, and in response to identified failure, a processor may initiate a mechanical sensing channel. Once initiated, the mechanical sensing channel may analyze an accelerometer signal to identify cardiac contractions. In some examples, other sensing channels may also analyze the accelerometer signal, e.g., to determine an activity level of the patient. For example, a sensing channel may analyze the accelerometer signal continuously to determine an activity level of the patient at all times. In this manner, the accelerometer may be turned on even when the mechanical sensing channel is not activated to identify cardiac contractions, and the mechanical sensing channel may selectively analyze the accelerometer signal to identify cardiac contractions in response to identifying a failure of the electrical sensing channel.

As described in more detail below, the sensing integrity module may be configured to identify a variety of mechanical and/or electrical failures of the electrical sensing channel. For example, the sensing integrity module may identify failures of one or more components of the electrical sensing channel. Mechanical and/or electrical failures of the electrical sensing channel may result in the absence of a signal and/or the presence of an inappropriate signal. Inappropriate signals may include, for example, frequencies outside of a physiological range, signals with high frequency and/or direct current input, and signals that exhibit railing, e.g., signals at, or alternating between maximum and positive and negative magnitudes. Example mechanical and/or electrical failures of the electrical sensing channel that may cause absent and/or inappropriate signals may include separation or detachment of one or more electrodes from tissue of the heart, a failure of a conductor connecting an electrode to sensing circuitry within a medical device, and other integrity issues. Examples of conductor failures may include broken conductors and/or shorted conductors. A processor may initiate the mechanical sensing channel in response to the identified failure of an electrical sensing channel, e.g., based on an absent and/or inappropriate signal.

Using the techniques of this disclosure, the mechanical sensing channel may allow a medical device to control delivery of therapeutic electrical signals to the heart based on sensed cardiac contractions, despite the failure of an electrical sensing channel. In medical devices that rely solely on electrical sensing, the medical device may determine that the sensed electrical signal is unreliable and provide a safety therapy, e.g., pacing pulses at a constant rate. As described in more detail below, the inclusion of a mechanical sensing channel may allow a medical device to deliver therapy that is better synchronized with the intrinsic rhythm of the heart, i.e., based on the mechanical rhythm of the heart, in these fault conditions.

As indicated above, once initiated, the mechanical sensing channel may analyze an accelerometer signal to identify cardiac contractions. The accelerometer may be positioned within or proximate to a heart of a patient such that it detects the rhythmic motion of one or more walls of the patient's heart. For example, the accelerometer may be positioned within an implantable medical device, such as a leadless pacemaker. A leadless pacemaker may be attached to a wall of the patient's heart, e.g., epicardially or endocardially. As another example, the accelerometer may be positioned within a lead, e.g., proximate to a distal end of a lead positioned within or outside a chamber of the heart. In general, the accelerometer may detect a signal indicative of the motion of the heart.

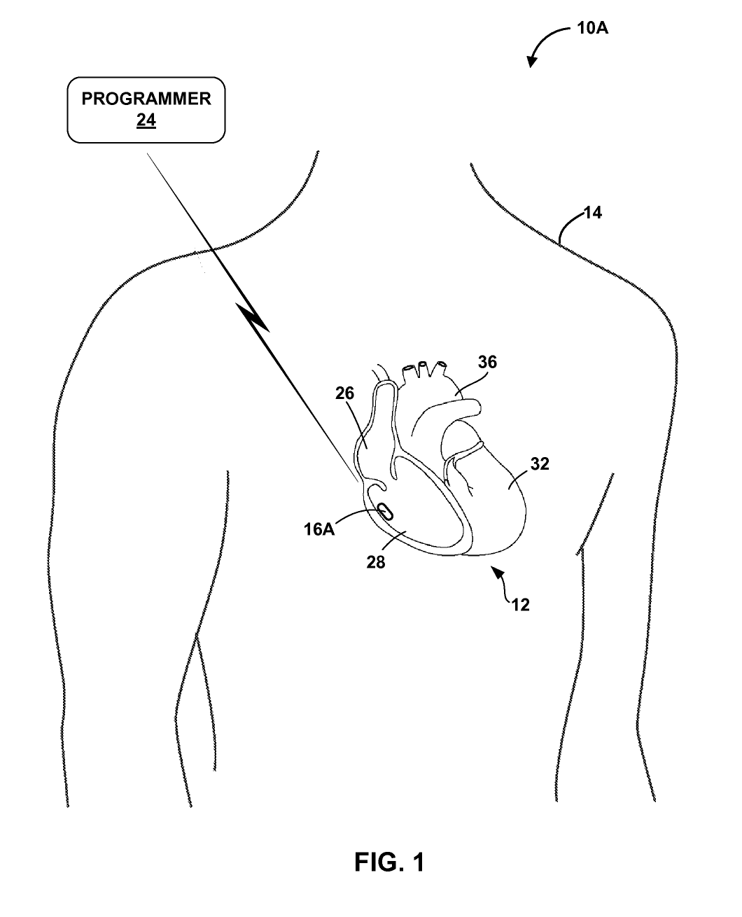

FIG. 1 is a conceptual diagram illustrating an example therapy system 10A that may be used to monitor one or more physiological parameters of patient 14 and/or to provide therapy to heart 12 of patient 14. Therapy system 10A includes an implantable medical device (IMD) 16A, which is coupled to programmer 24. IMD 16A may be an implantable leadless pacemaker that provides electrical signals to heart 12 via one or more electrodes (not shown in FIG. 1) on its outer housing. Additionally or alternatively, IMD 16A may sense electrical signals attendant to the depolarization and repolarization of heart 12 via electrodes on its outer housing. In some examples, IMD 16A provides pacing pulses to heart 12 based on the electrical signals sensed within heart 12. IMD 16A may also include an accelerometer (not shown in FIG. 1) within its housing. The accelerometer may detect an activity level of patient 14. Additionally or alternatively, as described in further detail below, the accelerometer may be utilized to identify cardiac contractions, e.g., in response to identifying the failure of an electrical sensing channel. Patient 14 is ordinarily, but not necessarily, a human patient.

In the example of FIG. 1, IMD 16A is positioned wholly within heart 12 proximate to an inner wall of right ventricle 28 to provide right ventricular (RV) pacing. Although IMD 16A is shown within heart 12 and proximate to an inner wall of right ventricle 28 in the example of FIG. 1, IMD 16A may be positioned at any other location outside or within heart 12. For example, IMD 16A may be positioned outside or within right atrium 26, left atrium 36, and/or left ventricle 32, e.g., to provide right atrial, left atrial, and left ventricular pacing, respectively. Depending in the location of implant, IMD 16A may include other stimulation functionalities. For example, IMD 16A may provide atrioventricular nodal stimulation, fat pad stimulation, vagal stimulation, or other types of neurostimulation. In other examples, IMD 16A may be a monitor that senses one or more parameters of heart 12 and may not provide any stimulation functionality. In some examples, system 10A may include a plurality of leadless IMDs 16A, e.g., to provide stimulation and/or sensing at a variety of locations.

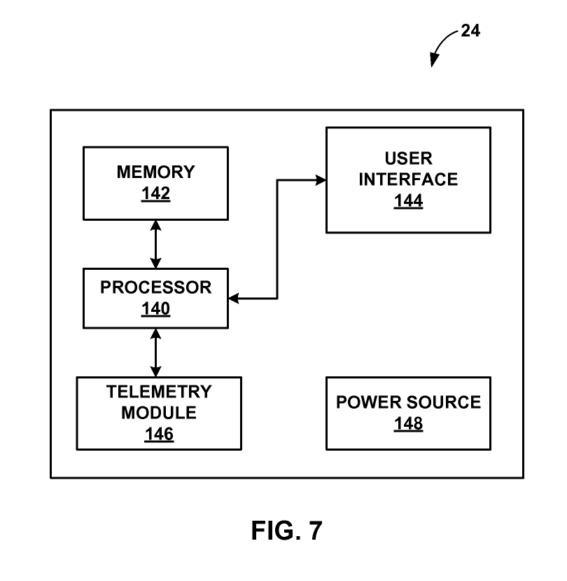

FIG. 1 further depicts programmer 24 in communication with IMD 16A. In some examples, programmer 24 comprises a handheld computing device, computer workstation, or networked computing device. Programmer 24, shown and described in more detail below with respect to FIG. 7, includes a user interface that presents information to and receives input from a user. It should be noted that the user may also interact with programmer 24 remotely via a networked computing device.

A user, such as a physician, technician, surgeon, electrophysiologist, other clinician, or patient, interacts with programmer 24 to communicate with IMD 16A. For example, the user may interact with programmer 24 to retrieve physiological or diagnostic information from IMD 16A. A user may also interact with programmer 24 to program IMD 16A, e.g., select values for operational parameters of the IMD 16A. For example, the user may use programmer 24 to retrieve information from IMD 16A regarding the rhythm of heart 12, trends therein over time, or arrhythmic episodes.

In some examples, the user of programmer 24 may receive an alert that a mechanical sensing channel has been activated to identify cardiac contractions in response to a detected failure of an electrical sensing channel. The alert may include an indication of the type of failure and/or confirmation that the mechanical sensing channel is detecting cardiac contractions. The alert may include a visual indication on a user interface of programmer 24. Additionally or alternatively, the alert may include vibration and/or audible notification.

As another example, the user may use programmer 24 to retrieve information from IMD 16A regarding other sensed physiological parameters of patient 14 or information derived from sensed physiological parameters, such intracardiac or intravascular pressure, activity, posture, respiration, tissue perfusion, heart sounds, cardiac electrogram (EGM), intracardiac impedance, or thoracic impedance. In some examples, the user may use programmer 24 to retrieve information from IMD 16A regarding the performance or integrity of IMD 16A or other components of system 10A, or a power source of IMD 16A. As another example, the user may interact with programmer 24 to program, e.g., select parameters for, therapies provided by IMD 16A, such pacing and, optionally, neurostimulation.

IMD 16A and programmer 24 may communicate via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the patient's body near the IMD 16A implant site in order to improve the quality or security of communication between IMD 16A and programmer 24.

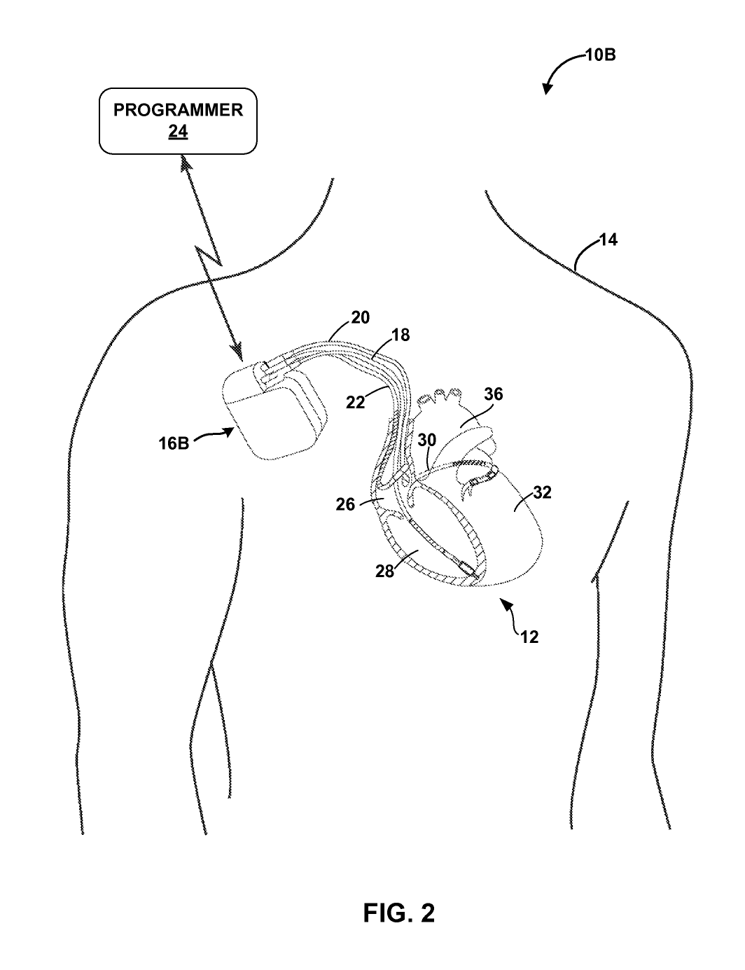

FIG. 2 is a conceptual diagram illustrating another example therapy system 10B that may be used to monitor one or more physiological parameters of patient 14 and/or to provide therapy to heart 12 of patient 14. Therapy system 10B includes IMD 16B, which is coupled to leads 18, 20, and 22, and programmer 24. In one example, IMD 16B may be an implantable pacemaker that provides electrical signals to heart 12 via electrodes coupled to one or more of leads 18, 20, and 22. In addition to pacing therapy, IMD 16B may deliver neurostimulation signals. In some examples, IMD 16B may also include cardioversion and/or defibrillation functionalities. In other examples, IMD 16B may not provide any stimulation functionalities and, instead, may be a dedicated monitoring device. Patient 14 is ordinarily, but not necessarily, a human patient.

Leads 18, 20, 22 extend into the heart 12 of patient 14 to sense electrical activity of heart 12 and/or deliver electrical stimulation to heart 12. In the example shown in FIG. 2, right ventricular (RV) lead 18 extends through one or more veins (not shown), the superior vena cava (not shown), right atrium 26, and into right ventricle 28. RV lead 18 may be used to deliver RV pacing to heart 12. Left ventricular (LV) lead 20 extends through one or more veins, the vena cava, right atrium 26, and into the coronary sinus 30 to a region adjacent to the free wall of left ventricle 32 of heart 12. LV lead 20 may be used to deliver LV pacing to heart 12. Right atrial (RA) lead 22 extends through one or more veins and the vena cava, and into the right atrium 26 of heart 12. RA lead 22 may be used to deliver RA pacing to heart 12.

In some examples, system 10B may additionally or alternatively include one or more leads or lead segments (not shown in FIG. 2) that deploy one or more electrodes within the vena cava or other vein, or within or near the aorta. Furthermore, in another example, system 10B may additionally or alternatively include one or more additional intravenous or extravascular leads or lead segments that deploy one or more electrodes epicardially, e.g., near an epicardial fat pad, or proximate to the vagus nerve. In other examples, system 10B need not include one of ventricular leads 18 and 20.

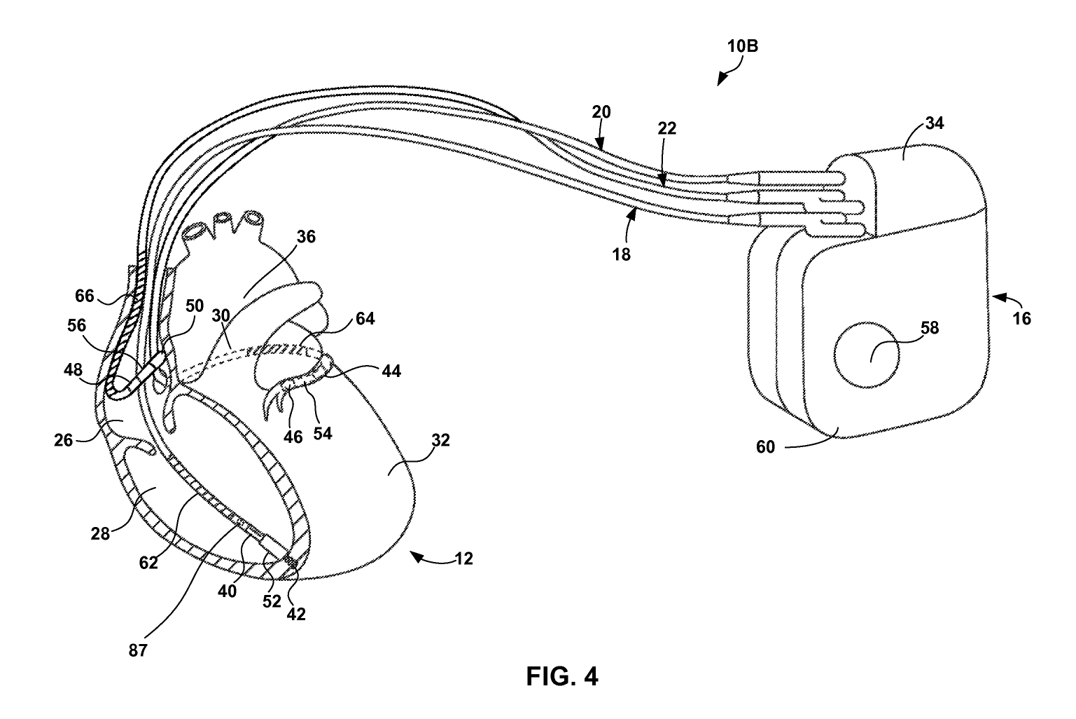

IMD 16B may sense electrical signals attendant to the depolarization and repolarization of heart 12 via electrodes (described in further detail with respect to FIG. 4) coupled to at least one of the leads 18, 20, 22. In some examples, IMD 16B provides pacing pulses to heart 12 based on the electrical signals sensed within heart 12. The configurations of electrodes used by IMD 16B for sensing and pacing may be unipolar or bipolar.

System 10B may also include an accelerometer (not shown in FIG. 2) proximate to a distal end of one of leads 18, 20, 22. For example, the accelerometer may be positioned proximate to a wall of heart 12 such that it detects the rhythmic motion of heart 12. Using the techniques of this disclosure, the accelerometer may be utilized to identify cardiac contractions, e.g., in response to identifying the failure of an electrical sensing channel, as described in further detail below. In some examples, the accelerometer may also be utilized to determine an activity level of patient 14.

IMD 16B may also provide neurostimulation therapy, defibrillation therapy and/or cardioversion therapy via electrodes located on at least one of the leads 18, 20, 22. For example, IMD 16B may deliver defibrillation therapy to heart 12 in the form of electrical pulses upon detecting ventricular fibrillation of ventricles 28 and 32. In some examples, IMD 16B may be programmed to deliver a progression of therapies, e.g., pulses with increasing energy levels, until a fibrillation of heart 12 is stopped. As another example, IMD 16B may deliver cardioversion or ATP in response to detecting ventricular tachycardia, such as tachycardia of ventricles 28 and 32.

As described above with respect to IMD 16A of FIG. 1, programmer 24 may also be used to communicate with IMD 16B. In addition to the functions described with respect to IMD 16A of FIG. 1, a user may use programmer 24 to retrieve information from IMD 16B regarding the performance or integrity of leads 18, 20 and 22 and may interact with programmer 24 to program, e.g., select parameters for, any additional therapies provided by IMD 16B, such as cardioversion and/or defibrillation.

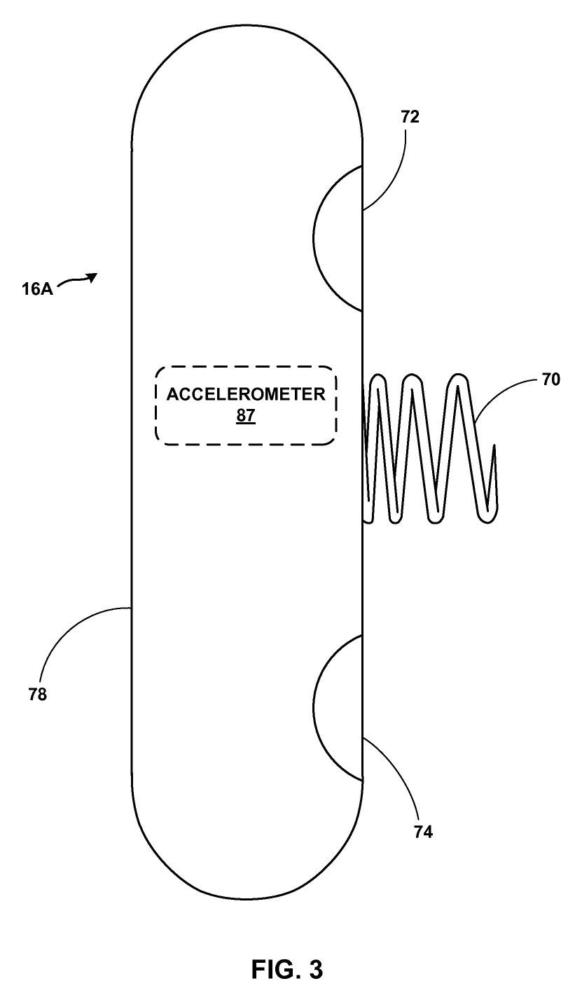

FIG. 3 is a conceptual diagram illustrating leadless IMD 16A of FIG. 1 in further detail. In the example of FIG. 3, leadless IMD 16A include fixation mechanism 70. Fixation mechanism 70 may anchor leadless IMD 16A to a wall of heart 12. For example, fixation mechanism 70 may take the form of a helical structure that may be screwed into a wall of heart 12. Alternatively, other structures of fixation mechanism 70, e.g., tines, adhesive, or sutures, may be utilized. In some examples, fixation mechanism is conductive and may be used as an electrode, e.g., to deliver therapeutic electrical signals to heart 12 and/or sense intrinsic depolarizations of heart 12.

Leadless IMD 16A may also include electrodes 72 and 74 on its outer housing 78. Electrodes 72 and 74 may be used to deliver therapeutic electrical signals to heart 12 and/or sense intrinsic depolarizations of heart 12. Electrodes 72 and 74 may be formed integrally with an outer surface of hermetically-sealed housing 78 of IMD 16A or otherwise coupled to housing 78. In this manner, electrodes 72 and 74 may be referred to as housing electrodes. In some examples, housing electrodes 72 and 74 are defined by uninsulated portions of an outward facing portion of housing 78 of IMD 16A. Other division between insulated and uninsulated portions of housing 78 may be employed to define a different number or configuration of housing electrodes. For example, in an alternative configuration, IMD 16A may include a single housing electrode that comprises substantially all of housing 78, and may be used in combination with an electrode formed by fixation mechanism 70 for sensing and/or delivery of therapy.

Leadless IMD 16A also includes accelerometer 87 within housing 78. When IMD 16A is anchored to or otherwise coupled to a wall of heart 12, IMD 16A may experience the motion of heart 12. Accelerometer 87 may detect cardiac contractions of heart 12 based on this motion. For example, accelerometer 87 may be a single axis accelerometer that detect motion, in this case motion of heart 12, along a single axis. As another example, accelerometer 87 may be a multi-axis detect motion along multiple axes, e.g., along three perpendicular axes. As yet another example, accelerometer 87 may include more than one accelerometer. As described in further detail below, accelerometer 87 may be used to identify cardiac contractions of heart 12 in response to identifying the failure of an electrical sensing channel. IMD 16A may generally control the delivery of therapeutic electrical stimulation based on the electrical depolarizations of heart 12 detected by an electrical sensing channel. Upon detecting a failure of the electrical sensing channel, IMD 16A may utilize the mechanical sensing channel to identify cardiac contractions and control delivery of therapeutic electrical stimulation based on the detected cardiac contractions. The inclusion of a mechanical sensing channel may allow a medical device to deliver therapy that is better synchronized with the intrinsic rhythm of the heart, i.e., based on the mechanical rhythm of the heart, in circumstances in which an electrical sensing channel fails. A mechanical sensing channel may also be used in cardiac monitoring devices in response to failure of an electrical sensing channel to allow the monitoring device to maintain continuous monitoring of the rhythm of heart 12.

FIG. 4 is a conceptual diagram illustrating IMD 16B and leads 18, 20, 22 of therapy system 10B of FIG. 2 in greater detail. Leads 18, 20, 22 may be electrically coupled to a signal generator and a sensing module of IMD 16B via connector block 34. In some examples, proximal ends of leads 18, 20, 22 may include electrical contacts that electrically couple to respective electrical contacts within connector block 34 of IMD 16B. In some examples, a single connector, e.g., an IS-4 or DF-4 connector, may connect multiple electrical contacts to connector block 34. In addition, in some examples, leads 18, 20, 22 may be mechanically coupled to connector block 34 with the aid of set screws, connection pins, snap connectors, or another suitable mechanical coupling mechanism.

Each of the leads 18, 20, 22 includes an elongated insulative lead body, which may carry a number of concentric coiled conductors separated from one another by tubular insulative sheaths. Bipolar electrodes 40 and 42 are located adjacent to a distal end of lead 18 in right ventricle 28. In addition, bipolar electrodes 44 and 46 are located adjacent to a distal end of lead 20 in left ventricle 32 and bipolar electrodes 48 and 50 are located adjacent to a distal end of lead 22 in right atrium 26. In the illustrated example, there are no electrodes located in left atrium 36. However, other examples may include electrodes in left atrium 36.

Electrodes 40, 44, and 48 may take the form of ring electrodes, and electrodes 42, 46, and 50 may take the form of extendable helix tip electrodes mounted retractably within insulative electrode heads 52, 54, and 56, respectively. In some examples, one or more of electrodes 42, 46, and 50 may take the form of pre-exposed helix tip electrodes. In other examples, one or more of electrodes 42, 46, and 50 may take the form of small circular electrodes at the tip of a tined lead or other fixation element. Leads 18, 20, 22 also include elongated electrodes 62, 64, 66, respectively, which may take the form of a coil. Each of the electrodes 40, 42, 44, 46, 48, 50, 62, 64, and 66 may be electrically coupled to a respective one of the coiled conductors within the lead body of its associated lead 18, 20, 22, and thereby coupled to respective ones of the electrical contacts on the proximal end of leads 18, 20, 22.

In some examples, as illustrated in FIG. 4, IMD 16B includes one or more housing electrodes, such as housing electrode 58, which may be formed integrally with an outer surface of hermetically-sealed housing 60 of IMD 16B or otherwise coupled to housing 60. In some examples, housing electrode 58 is defined by an uninsulated portion of an outward facing portion of housing 60 of IMD 16B. Other division between insulated and uninsulated portions of housing 60 may be employed to define two or more housing electrodes. In some examples, housing electrode 58 comprises substantially all of housing 60.

IMD 16B may sense electrical signals attendant to the depolarization and repolarization of heart 12 via electrodes 40, 42, 44, 46, 48, 50, 58, 62, 64, and 66. The electrical signals are conducted to IMD 16B from the electrodes via conductors within the respective leads 18, 20, 22 or, in the case of housing electrode 58, a conductor coupled to housing electrode 58. IMD 16B may sense such electrical signals via any bipolar combination of electrodes 40, 42, 44, 46, 48, 50, 58, 62, 64, and 66. Furthermore, any of the electrodes 40, 42, 44, 46, 48, 50, 58, 62, 64, and 66 may be used for unipolar sensing in combination with housing electrode 58.

In some examples, IMD 16B delivers pacing pulses via bipolar combinations of electrodes 40, 42, 44, 46, 48 and 50 to produce depolarization of cardiac tissue of heart 12. In some examples, IMD 16B delivers pacing pulses via any of electrodes 40, 42, 44, 46, 48 and 50 in combination with housing electrode 58 in a unipolar configuration.

Furthermore, IMD 16B may deliver defibrillation pulses to heart 12 via any combination of elongated electrodes 62, 64, 66, and housing electrode 58. Electrodes 58, 62, 64, 66 may also be used to deliver cardioversion pulses to heart 12. Electrodes 62, 64, 66 may be fabricated from any suitable electrically conductive material, such as, but not limited to, platinum, platinum alloy or other materials known to be usable in implantable defibrillation electrodes.

One or more of leads 18, 20, and 22 may also include an accelerometer 87 positioned proximate to its distal end. As one example, accelerometer 87 may be positioned within the lead body of LV lead 18. For example, accelerometer 87 is depicted near the distal end of LV lead 18 in FIG. 4. One or more accelerometers positioned proximate to the distal end of one or more of leads 18, 20, and 22 may experience the motion of heart 12. As described in further detail below, an accelerometer signal may be analyzed to identify cardiac contractions of heart 12 in response to identifying the failure of an electrical sensing channel. IMD 16B may generally control the delivery of therapeutic electrical stimulation based on the electrical depolarizations of heart 12 detected by an electrical sensing channel. Upon detecting a failure of the electrical sensing channel, IMD 16B may utilize the mechanical sensing channel to identify cardiac contractions and control delivery of therapeutic electrical stimulation based on the detected cardiac contractions. The inclusion of a mechanical sensing channel may allow a medical device to deliver therapy that is better synchronized with the intrinsic rhythm of the heart, i.e., based on the mechanical rhythm of the heart, in circumstances in which an electrical sensing channel fails. A mechanical sensing channel may also be used in cardiac monitoring devices in response to failure of an electrical sensing channel to allow the monitoring device to maintain continuous monitoring of the rhythm of heart 12.

The configuration of system 10B illustrated in FIGS. 2 and 4 is merely one example. In other examples, a system may include epicardial leads and/or patch electrodes instead of or in addition to the transvenous leads 18, 20, 22 illustrated in FIG. 2. Further, IMD 16B need not be implanted within patient 14. In examples in which IMD 16B is not implanted in patient 14, IMD 16B may deliver defibrillation pulses and other therapies to heart 12 via percutaneous leads that extend through the skin of patient 14 to a variety of positions within or outside of heart 12.

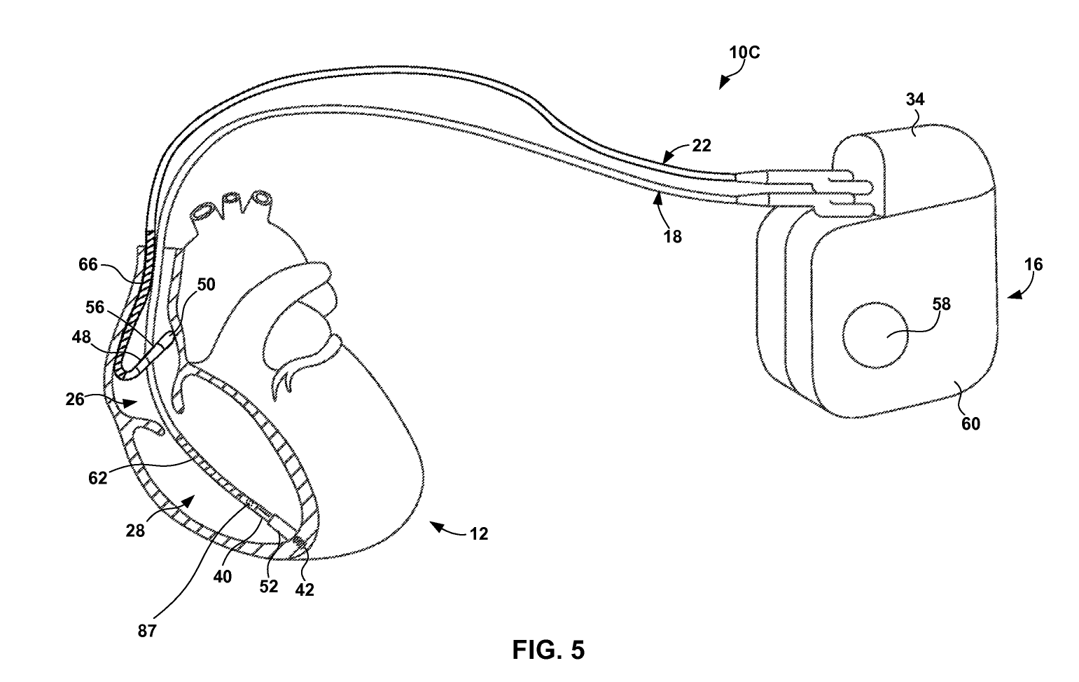

In addition, in other examples, a system may include any suitable number of leads coupled to IMD 16B, and each of the leads may extend to any location within or proximate to heart 12. For example, other examples of systems may include three transvenous leads located as illustrated in FIGS. 2 and 4, and an additional lead located within or proximate to left atrium 36. Other examples of systems may include a single lead that extends from IMD 16B into right atrium 26 or right ventricle 28, or two leads that extend into a respective one of the right ventricle 26 and right atrium 26. An example of this type of system is shown in FIG. 5. Any electrodes located on these additional leads may be used in sensing and/or stimulation configurations.

FIG. 5 is a conceptual diagram illustrating another example system 10C, which is similar to system 10B of FIGS. 2 and 4, but includes two leads 18, 22, rather than three leads. Leads 18, 22 are implanted within right ventricle 28 and right atrium 26, respectively. System 10C shown in FIG. 5 may be useful for physiological sensing and/or providing pacing, cardioversion, or other therapies to heart 12. As described with respect to system 10B of FIGS. 2 and 4, one or both of leads 18 and 22 may include an accelerometer positioned proximate to its distal end that may be used to detect cardiac contractions in response to identifying a failure of an electrical sensing channel. For example, accelerometer 87 is depicted proximate to the distal end of lead 18 in the example of FIG. 5.

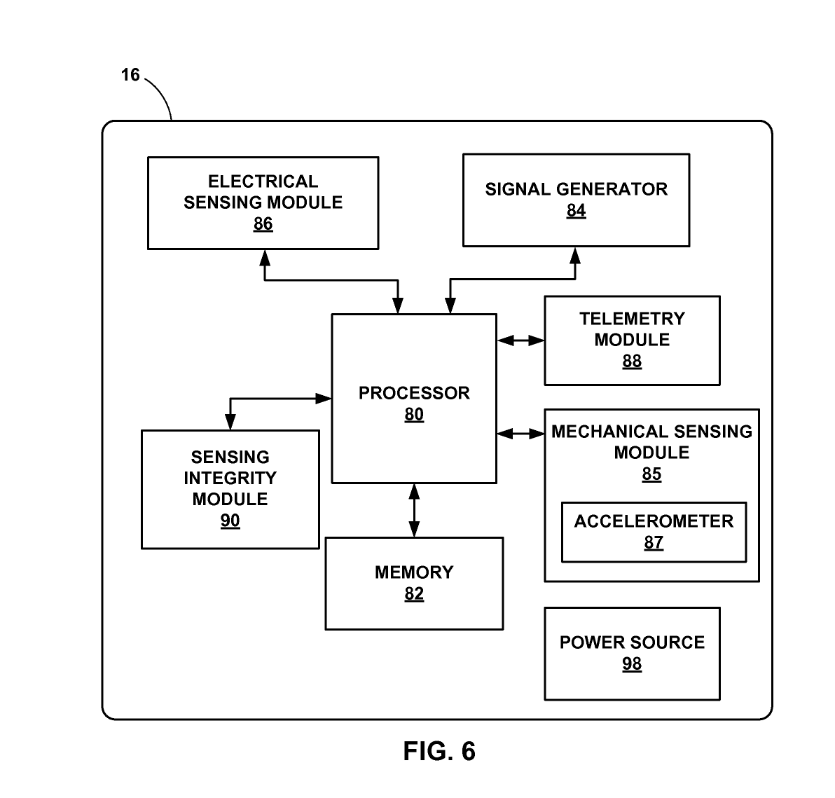

FIG. 6 is a functional block diagram illustrating one example configuration of IMD 16A of FIGS. 1 and 3 or IMD 16B of FIGS. 2, 4, and 5 (referred to generally as IMD 16). In the example illustrated by FIG. 6, IMD 16 includes a processor 80, memory 82, signal generator 84, mechanical sensing module 85, electrical sensing module 86, accelerometer 87, telemetry module 88, and power source 98. Memory 82 may include computer-readable instructions that, when executed by processor 80, cause IMD 16 and processor 80 to perform various functions attributed to IMD 16 and processor 80 herein. Memory 82 may be a computer-readable storage medium, including any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media.

Processor 80 may include any one or more of a microprocessor, a controller, a digital signal processor (DSP), an application specific integrated circuit (ASIC), a field-programmable gate array (FPGA), or equivalent discrete or integrated logic circuitry. In some examples, processor 80 may include multiple components, such as any combination of one or more microprocessors, one or more controllers, one or more DSPs, one or more ASICs, or one or more FPGAs, as well as other discrete or integrated logic circuitry. The functions attributed to processor 80 in this disclosure may be embodied as software, firmware, hardware or any combination thereof. IMD 16 also includes a sensing integrity module 90, as illustrated in FIG. 6, which may be implemented by processor 80, e.g., as a hardware component of processor 80, or a software component executed by processor 80.

Processor 80 controls signal generator 84 to deliver stimulation therapy to heart 12 according to operational parameters or programs, which may be stored in memory 82. For example, processor 80 may control signal generator 84 to deliver electrical pulses with the amplitudes, pulse widths, frequency, or electrode polarities specified by the selected one or more therapy programs.

Signal generator 84, as well as electrical sensing module 86, is electrically coupled to electrodes of IMD 16 and/or leads coupled to IMD 16. In the example of IMD 16A of FIG. 3, signal generator 84 and electrical sensing module 86 are coupled to electrodes 72 and 74, e.g., via conductors disposed within housing 78 of IMD 16A. In examples in which fixation mechanism 70 functions as an electrode, signal generator 84 and electrical sensing module 86 may also be coupled to fixation mechanism 70, e.g., via a conductor disposed within housing 78 of IMD 16A. In the example of IMD 16B of FIG. 4, signal generator 84 and electrical sensing module 86 are coupled to electrodes 40, 42, 44, 46, 48, 50, 58, 62, 64, and 66, e.g., via conductors of the respective lead 18, 20, 22, or, in the case of housing electrode 58, via an electrical conductor disposed within housing 60 of IMD 16B.

In the example illustrated in FIG. 6, signal generator 84 is configured to generate and deliver electrical stimulation therapy to heart 12. For example, signal generator 84 may deliver pacing, cardioversion, defibrillation, and/or neurostimulation therapy via at least a subset of the available electrodes. In some examples, signal generator 84 delivers one or more of these types of stimulation in the form of electrical pulses. In other examples, signal generator 84 may deliver one or more of these types of stimulation in the form of other signals, such as sine waves, square waves, or other substantially continuous time signals.

Signal generator 84 may include a switch module and processor 80 may use the switch module to select, e.g., via a data/address bus, which of the available electrodes are used to deliver stimulation signals, e.g., pacing, cardioversion, defibrillation, and/or neurostimulation signals. The switch module may include a switch array, switch matrix, multiplexer, or any other type of switching device suitable to selectively couple a signal to selected electrodes.

Electrical sensing module 86 monitors signals from at least a subset of the available electrodes in order to monitor electrical activity of heart 12. Electrical sensing module 86 may also include a switch module to select which of the available electrodes are used to sense the heart activity. In some examples, processor 80 may select the electrodes that function as sense electrodes, i.e., select the sensing configuration, via the switch module within electrical sensing module 86, e.g., by providing signals via a data/address bus.

In some examples, electrical sensing module 86 includes multiple detection channels, each of which may comprise an amplifier. Each sensing channel may detect electrical activity in respective chambers of heart 12, and may be configured to detect either R-waves or P-waves. In some examples, electrical sensing module 86 or processor 80 may include an analog-to-digital converter for digitizing the signal received from a sensing channel for electrogram (EGM) signal processing by processor 80. In response to the signals from processor 80, the switch module within electrical sensing module 86 may couple the outputs from the selected electrodes to one of the detection channels or the analog-to-digital converter.

During pacing, escape interval counters maintained by processor 80 may be reset upon sensing of R-waves and P-waves with respective detection channels of electrical sensing module 86. Signal generator 84 may include pacer output circuits that are coupled, e.g., selectively by a switching module, to any combination of the available electrodes appropriate for delivery of a bipolar or unipolar pacing pulse to one or more of the chambers of heart 12. Processor 80 may control signal generator 84 to deliver a pacing pulse to a chamber upon expiration of an escape interval. Processor 80 may reset the escape interval counters upon the generation of pacing pulses by signal generator 84, or detection of an intrinsic depolarization in a chamber, and thereby control the basic timing of cardiac pacing functions. The escape interval counters may include P-P, V-V, RV-LV, A-V, A-RV, or A-LV interval counters, as examples. The value of the count present in the escape interval counters when reset by sensed R-waves and P-waves may be used by processor 80 to measure the durations of R-R intervals, P-P intervals, P-R intervals and R-P intervals. Processor 80 may use the count in the interval counters to detect heart rate, such as an atrial rate or ventricular rate.

IMD 16 also includes sensing integrity module 90. Sensing integrity module 90 may identify failures of the detection channels of electrical sensing module 86. For example, sensing integrity module 90 may monitor, e.g., periodically or continuously, one or more signals from electrical sensing module 86. Sensing integrity module 90 may be configured to identify a variety of mechanical and/or electrical failures of one or more channels of electrical sensing module 86. For example, sensing integrity module 90 may identify failures of one or more components, e.g., conductors or electrodes, of an electrical sensing channel. Mechanical and/or electrical failures of the electrical sensing channel may result in the absence of a signal and/or the presence of an inappropriate signal. Inappropriate signals may include frequencies outside of a physiological range, signals with high frequency and/or direct current input, and signals that exhibit railing. In some example implementations, sensing integrity module includes one or more filters for filtering a signal received by an electrical sensing channel in order to filter out frequencies outside of a physiological range, e.g., noise. Additionally or alternatively, electrical sensing module 86 may include one or more filters for filtering a signal received by an electrical sensing channel in order to filter out frequencies outside of a physiological range, e.g., noise. Example mechanical and/or electrical failures of the electrical sensing channel that may cause absence and/or inappropriate signals may include, for example, separation or detachment of one or more electrodes from tissue of the heart, failure of a conductor connecting an electrode to electrical sensing module 86, and other integrity issues. Examples of conductor failures may include broken conductors and/or shorted conductors.

Sensing integrity module 90 may, e.g., periodically or continuously, evaluate signals sensed by electrical sensing module 86. For example, sensing integrity module 90 may identify inappropriate signal characteristics, e.g., lack of signal, low signal amplitudes below a threshold at which electrical sensing module 86 may detect cardiac depolarizations or other cardiac events, frequencies outside of a physiological range, signals with high frequency and/or direct current input, and signals that exhibit railing, to identify failure of an electrical sensing channel. In some examples, sensing integrity module 90 may measure the impedance along an electrical signal channel to identify failure of an electrical sensing channel.

As one example, if electrode 72 of IMD 16A (FIG. 3) separates from the tissue of heart 12, electrical sensing module 86 may not be able to detect the electrical depolarizations of heart 12 using the electrical sensing channel that includes electrode 72. Sensing integrity module 90 may detect the separation of electrode 72 from the tissue of heart 12 by identifying the absence of a signal, e.g., no signal of sufficient amplitude for detection in the frequency range associated with cardiac depolarizations, from the electrical sensing channel. In response to detecting the failure, processor 80 may initiate a mechanical sensing channel of mechanical sensing module 85 to identify cardiac contractions.

As another example, if a conductor of lead 18 that connects electrode 42 (FIG. 4) to electrical sensing module 86 is experiencing intermittent disconnection, electrical sensing module 86 may not be able to reliably capture the electrical depolarizations of heart 12 using the electrical sensing channel that includes electrode 42. Sensing integrity module 90 may detect the intermittent disconnection by identifying high frequency noise outside of the frequency range of physiological activity. In particular, sensing integrity module 90 may be configured to identify the high frequency noise associated with the “make/break” events resulting from intermittent fracture or disconnection of a conductor. In response to detecting the failure, processor 80 may initiate a mechanical sensing channel of mechanical sensing module 85 to identify cardiac contractions.

In response to detecting a failure, processor 80 may initiate a mechanical sensing channel of mechanical sensing module 85 to identify cardiac contractions.

Mechanical sensing module 85 includes a channel configured to detect cardiac contractions. For example, mechanical sensing module 85 may analyze a signal generated by accelerometer 87. In some examples, mechanical sensing module 85 may include a bandpass filter configured to pass frequencies associated with heart rate information and attenuate frequencies non-physiological signals, e.g., signals associated with patient movement, and may detect cardiac contractions using the filtered signal. Accelerometer 87 may be positioned such that it experiences the rhythmic motion of heart 12.

Using various techniques of this disclosure, IMD 16 may detect arrhythmias based on the filtered accelerometer signal. For example, a bandpass filter of mechanical sensing module 85 may be configured to filter out frequencies of a signal generated by accelerometer 87 that are not within a range of physiological frequencies. Processor 80 may analyze the filtered accelerometer signal and, if the signal is at a high end of a range of physiological frequencies, then processor 80 may determine that the patient is experiencing ventricular tachycardia or ventricular fibrillation. If the signal is at a low end of a range of physiological frequencies, then processor 80 may determine that the patient is be experiencing bradycardia.

Although accelerometer 87 is illustrated within IMD 16 in the example of FIG. 6, in some examples accelerometer 87 may be positioned outside of the housing of IMD 16. As one example, as described with respect to FIG. 4, an accelerometer may be position proximate to a distal end of a lead.

In some examples, mechanical sensing module 85 may include multiple channels. By way of specific example, mechanical sensing module 85 may include one channel for identifying cardiac contractions and another channel for identifying an activity level of the patient via a signal generated by accelerometer 87. Processor 80 may independently activate the various channels of mechanical sensing module 85. In this manner, mechanical sensing module 85 may detect an activity level of the patient regardless of whether the channel for identifying cardiac contractions is activated. In some examples, mechanical sensing module 85 may continuously monitor an activity level of the patient and may selectively monitor cardiac contractions in response to sensing integrity module 90 identifying a failure of an electrical sensing channel of electrical sensing module 86. Selectively utilizing mechanical sensing module 85 to monitor cardiac contractions in response to identifying a failure of an electrical sensing channel may conserve power.

Telemetry module 88 includes any suitable hardware, firmware, software or any combination thereof for communicating with another device, such as programmer 24 (FIGS. 1 and 2). Under the control of processor 80, telemetry module 88 may receive downlink telemetry from and send uplink telemetry to programmer 24 with the aid of an antenna, which may be internal and/or external. Processor 80 may provide the data to be uplinked to programmer 24 and receive downlinked data from programmer 24 via an address/data bus. In some examples, telemetry module 88 may provide received data to processor 80 via a multiplexer.

In some examples, processor 80 may transmit an alert that a mechanical sensing channel has been activated to identify cardiac contractions to programmer 24 or another computing device via telemetry module 88 in response to a detected failure of an electrical sensing channel. The alert may include an indication of the type of failure and/or confirmation that the mechanical sensing channel is detecting cardiac contractions. The alert may include a visual indication on a user interface of programmer 24. Additionally or alternatively, the alert may include vibration and/or audible notification. Processor 80 may also transmit data associated with the detected failure of the electrical sensing channel, e.g., the time that the failure occurred, impedance data, and/or the inappropriate signal indicative of the detected failure.

FIG. 7 is a functional block diagram of an example configuration of programmer 24. As shown in FIG. 7, programmer 24 includes processor 140, memory 142, user interface 144, telemetry module 146, and power source 148. Programmer 24 may be a dedicated hardware device with dedicated software for programming of IMD 16. Alternatively, programmer 24 may be an off-the-shelf computing device running an application that enables programmer 24 to program IMD 16.

A user may use programmer 24 to select therapy programs (e.g., sets of stimulation parameters), generate new therapy programs, or modify therapy programs for IMD 16. The clinician may interact with programmer 24 via user interface 144, which may include a display to present a graphical user interface to a user, and a keypad or another mechanism for receiving input from a user.

Processor 140 can take the form one or more microprocessors, DSPs, ASICs, FPGAs, programmable logic circuitry, or the like, and the functions attributed to processor 140 in this disclosure may be embodied as hardware, firmware, software or any combination thereof. Memory 142 may store instructions and information that cause processor 140 to provide the functionality ascribed to programmer 24 in this disclosure. Memory 142 may include any fixed or removable magnetic, optical, or electrical media, such as RAM, ROM, CD-ROM, hard or floppy magnetic disks, EEPROM, or the like. Memory 142 may also include a removable memory portion that may be used to provide memory updates or increases in memory capacities. A removable memory may also allow patient data to be easily transferred to another computing device, or to be removed before programmer 24 is used to program therapy for another patient. Memory 142 may also store information that controls therapy delivery by IMD 16, such as stimulation parameter values.

Programmer 24 may communicate wirelessly with IMD 16, such as using RF communication or proximal inductive interaction. This wireless communication is possible through the use of telemetry module 146, which may be coupled to an internal antenna or an external antenna. An external antenna that is coupled to programmer 24 may correspond to the programming head that may be placed over heart 12, as described above with reference to FIG. 1. Telemetry module 146 may be similar to telemetry module 88 of IMD 16 (FIG. 6).

Telemetry module 146 may also be configured to communicate with another computing device via wireless communication techniques, or direct communication through a wired connection. Examples of local wireless communication techniques that may be employed to facilitate communication between programmer 24 and another computing device include RF communication according to the 802.11 or Bluetooth specification sets, infrared communication, e.g., according to the IrDA standard, or other standard or proprietary telemetry protocols. In this manner, other external devices may be capable of communicating with programmer 24 without needing to establish a secure wireless connection. An additional computing device in communication with programmer 24 may be a networked device such as a server capable of processing information retrieved from IMD 16.

In some examples, processor 140 of programmer 24 and/or one or more processors of one or more networked computers may perform all or a portion of the techniques described in this disclosure with respect to processor 80 and IMD 16. For example, processor 140 or another processor may receive one or more signals from electrical sensing module 86, a signal from accelerometer 87, or information regarding sensed parameters from IMD 16 via telemetry module 146. In some examples, processor 140 may process or analyze sensed signals, as described in this disclosure with respect to IMD 16 and processor 80. In some examples, processor 140 may include or implement sensing integrity module 90 to perform the techniques described in this disclosure with respect to sensing integrity module 90.

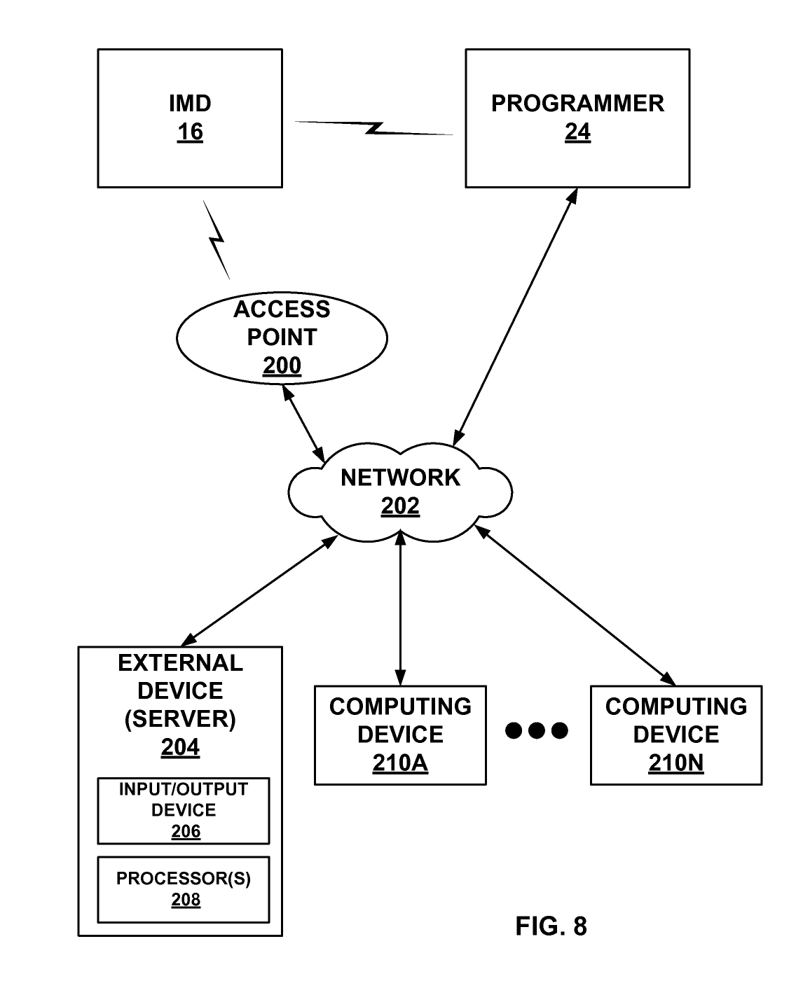

FIG. 8 is a block diagram illustrating an example system that includes an external device, such as a server 204, and one or more computing devices 210A-210N, that are coupled to the IMD 16 and programmer 24 (shown in FIGS. 1 and 2) via a network 202. In this example, IMD 16 may use its telemetry module 88 to communicate with programmer 24 via a first wireless connection, and to communication with an access point 200 via a second wireless connection. In the example of FIG. 8, access point 200, programmer 24, server 204, and computing devices 210A-210N are interconnected, and able to communicate with each other, through network 202. In some cases, one or more of access point 200, programmer 24, server 204, and computing devices 210A-210N may be coupled to network 202 through one or more wireless connections. IMD 16, programmer 24, server 204, and computing devices 210A-210N may each comprise one or more processors, such as one or more microprocessors, DSPs, ASICs, FPGAs, programmable logic circuitry, or the like, that may perform various functions and operations, such as those described herein.

Access point 200 may comprise a device that connects to network 202 via any of a variety of connections, such as telephone dial-up, digital subscriber line (DSL), or cable modem connections. In other examples, access point 200 may be coupled to network 202 through different forms of connections, including wired or wireless connections. In some examples, access point 200 may be co-located with patient 14 and may comprise one or more programming units and/or computing devices (e.g., one or more monitoring units) that may perform various functions and operations described herein. For example, access point 200 may include a home-monitoring unit that is co-located with patient 14 and that may monitor the activity of IMD 16. In some examples, server 204 or computing devices 210 may control or perform any of the various functions or operations described herein, e.g., include or implement sensing integrity module 90 and/or initiate a mechanical sensing channel in response to a detecting a failure of an electrical sensing channel.

In some cases, server 204 may be configured to provide a secure storage site for data that has been collected from IMD 16 and/or programmer 24. Network 202 may comprise a local area network, wide area network, or global network, such as the Internet. In some cases, programmer 24 or server 206 may assemble data in web pages or other documents for viewing by trained professionals, such as clinicians, via viewing terminals associated with computing devices 210A-210N. The illustrated system of FIG. 8 may be implemented, in some aspects, with general network technology and functionality similar to that provided by the Medtronic CareLink® Network developed by Medtronic, Inc., of Minneapolis, Minn.

In some examples, processor(s) 208 of server 204 may be configured to provide some or all of the functionality ascribed to IMD 16 and processor 80 herein. For example, processor 208 may receive one or more signals from electrical sensing module 86 or other information regarding sensed parameters from IMD 16 via access point 200 or programmer 24 and network 202. Processor 208 may also identify failures of electrical sensing channels based on the received signals. In some examples, server 204 relays received signals provided by one or more of IMD 16 or programmer 24 to one or more of computing devices 210 via network 202. A processor of a computing device 210 may provide some or all of the functionality ascribed to IMD 16 and processor 80 in this disclosure. In some examples, a processor of computing device 210 may include or implement sensing integrity module 90 to perform the techniques described in this disclosure with respect to sensing integrity module 90.

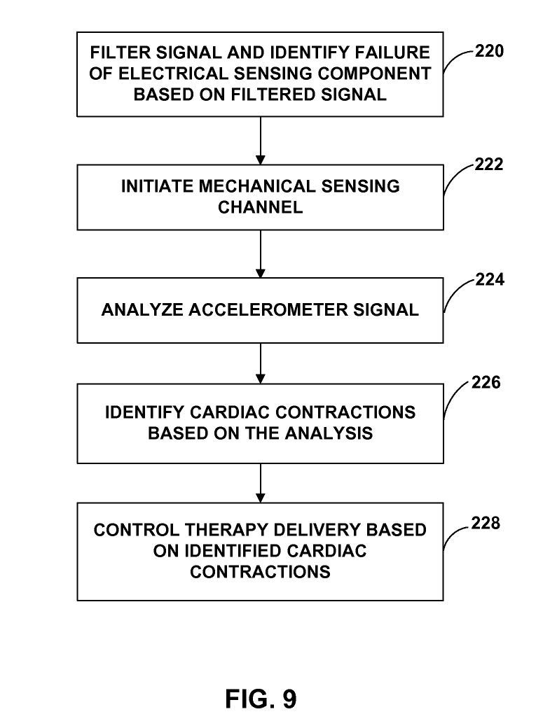

FIG. 9 is a flow diagram of an example method of using an accelerometer to identify cardiac contractions in response to detecting the failure of an electrical sensing channel. The example method of FIG. 9 is described as being performed by processor 80 and sensing integrity module 90 of IMD 16. In other examples, one or more other processors of one or more other devices may implement all or part of this method, e.g., may include or implement sensing integrity module 90.

Sensing integrity module 90 (and/or electrical sensing module 86) filters a signal received by an electrical sensing channel of IMD 16 and identifies the failure of an electrical sensing channel of electrical sensing module 86 based on the filtered signal (220). For example, sensing integrity module 90 may monitor, e.g., periodically or continuously, a signal from electrical sensing module 86. Sensing integrity module 90 may be configured to identify a variety of failures of one or more electrical sensing channels of electrical sensing module 86. For example, sensing integrity module 90 may identify mechanical and/or electrical failures. These failures may result in the absence of a signal and/or the presence of an inappropriate signal. Inappropriate signals may include, for example, frequencies outside of a physiological range, signals with high frequency and/or direct current input, and signals the exhibit railing. Some causes of such absent and/or inappropriate signals may include, for example, separation of an electrode from tissue, failure of a conductor connecting an electrode to electrical sensing module 86, and other integrity issues.

In response to the detected failure, processor 80 may initiate a mechanical sensing channel of mechanical sensing module 85 to identify cardiac contractions (222). The mechanical sensing channel may analyze a signal from accelerometer 87 (224) and identify cardiac contractions based on the analysis (226). For example, mechanical sensing module 85 may include a bandpass filter configured to pass frequencies associated with heart rate information and attenuate frequencies associated with patient movement.

In some example, mechanical sensing module 85 may include multiple channels. For example, mechanical sensing module 85 may include one channel for identifying cardiac contractions and another for identifying an activity level of the patient. These channels may be independently activated. In this manner, mechanical sensing module 85 may detect an activity level of the patient regardless of whether the channel for identifying cardiac contractions is activated. In some examples, mechanical sensing module 85 may continuously monitor an activity level of the patient and may selectively monitor cardiac contractions in response to sensing integrity module 90 identifying a failure of an electrical sensing channel of electrical sensing module 86.

Processor 80 may control signal generator 84 to deliver therapy based on the cardiac contractions detected using mechanical sensing module 85 (228). For example, processor 80 may rely on the cardiac contractions sensed via mechanical sensing module 85 to maintain an escape interval counter and control signal generator 84 to deliver a pacing pulse to a chamber of heart 12 upon expiration of an escape interval. In this manner, processor 80 may control the timing of pacing pulses based on cardiac contractions detected using mechanical sensing module.

Various examples of the disclosure have been described. These and other examples are within the scope of the following claims.

1. A method comprising:
filtering a signal received by an electrical sensing channel of an implantable medical device (IMD) configured to detect electrical depolarizations of a heart of a patient;
identifying a failure of the electrical sensing channel of the IMD based on the filtered signal; and
in response to identifying the failure, initiating a mechanical sensing channel of the implantable medical device to identify mechanical cardiac contractions.

2. The method of claim 1, wherein the mechanical sensing channel analyzes a signal from at least one accelerometer to identify the mechanical cardiac contractions.

3. The method of claim 1, wherein identifying the failure comprises identifying a detachment of an electrode of the electrical sensing channel from a tissue of the patient.

4. The method of claim 1, wherein identifying the failure comprises identifying a failure of a conductor of the electrical sensing channel.

5. The method of claim 1, further comprising controlling delivery of therapeutic electrical stimulation to the patient based on the identified mechanical cardiac contractions.

6. The method of claim 5, wherein the therapeutic electrical stimulation comprises pacing of the heart of the patient.

7. The method of claim 1, further comprising generating an alert in response to the initiation of the mechanical sensing channel.

8. A system comprising:
an accelerometer positioned proximate to a wall of a heart of a patient;
an electrical sensing channel configured to detect electrical depolarizations of the heart of the patient;
a mechanical sensing channel configured to analyze a signal from the accelerometer to identify mechanical contractions of the heart of the patient;
a sensing integrity module configured to:
filter a signal received by the electrical sensing channel; and
identify a failure of the electrical sensing channel based on the filtered signal; and
a processor configured to initiate the mechanical sensing channel in response to the identified failure.

9. The system of claim 8, wherein the electrical sensing channel comprises an electrode positioned proximate to the heart of the patient, sensing circuitry, and a conductor that connects the electrode to the sensing circuitry.

10. The system of claim 9, wherein the sensing integrity module identifies the failure by identifying a detachment of the electrode of the electrical sensing channel from a tissue of the patient.

11. The system of claim 9, wherein the sensing integrity module identifies the failure by identifying a failure of a conductor of the electrical sensing channel.

12. The system of claim 8, further comprising a signal generator configured to deliver therapeutic electrical stimulation to the patient, wherein the processor controls the signal generator to deliver the therapeutic electrical stimulation based on the identified mechanical cardiac contractions.

13. The system of claim 12, wherein the signal generator is configured to deliver pacing therapy to the heart of the patient.

14. The system of claim 8, further comprising a programmer, the programmer including a user interface.

15. The system of claim 14, wherein the user interface is configured to provide an alert in response to the processor initiating the mechanical sensing channel.

16. The system of claim 8, further comprising an implantable medical device, wherein the implantable medical device comprises the electrical sensing channel, the mechanical sensing channel, and the processor.

17. The system of claim 16, wherein the implantable medical device further comprises the sensing integrity module.

18. The system of claim 16, wherein the implantable medical device comprises a leadless pacemaker, and wherein the implantable medical device includes the accelerometer.

19. A computer-readable storage medium comprising instructions that, when executed, cause a programmable processor to:
filter a signal received by an electrical sensing channel of an implantable medical device (IMD) configured to detect electrical depolarizations of a heart of a patient;
identify a failure of the electrical sensing channel of the IMD based on the filtered signal; and
in response to identifying the failure, initiating a mechanical sensing channel to identify mechanical cardiac contractions.

20. A system comprising:
means for filtering a signal received by an electrical sensing channel of an implantable medical device (IMD) configured to detect electrical depolarizations of a heart of a patient;
means for identifying a failure of the electrical sensing channel of the IMD based on the filtered signal; and
means for initiating a mechanical sensing channel to identify mechanical cardiac contractions in response to identifying the failure.
